(19) United States
(12) Patent Application Publication
(10) Pub. No.: US 2005/0203122 A1
(43) Pub. Date: Sep. 15, 2005

(54) Benzenesulfonic acid salts of clopidogrel, methods for preparing same, and pharmaceutical formulations thereof

(75) Inventors: Stephen M. Doser, Nordheide (DE); Klaus Glanzer, Hamburg (DE)

Correspondence Address: Kevin C. Hooper, Esq.;BRYAN CAVE LLP, 33rd Floor, 1290 Avenue of the Americas, New York, NY, 10104, US

(73) Assignee: Helm AG

(21) Appl. No.: 11/113,467

(22) Filed: Apr. 25, 2005

Related U.S. Application Data
(63) Continuation-in-part of application No. 10/498,860.

(30) Foreign Application Priority Data
Feb. 13, 2003 (DE) 103 05 984.9

(51) Int. Cl. A61K 31/4743; C07D 498/02

(52) U.S. Cl. 514/301; 546/114

(57) ABSTRACT

The present invention relates to benzenesulfonic acid salts of clopidogrel, including crystalline clopidogrel besylate in form III, processes for the preparation of these salts, the use of these salts for producing pharmaceutical formulations, and to pharmaceutical compositions comprising these salts.

This Application is a Continuation-In-Part of prior application Ser. No. 10/498,860, which is the National Stage of International Application PCT/EP2004/001369, filed Feb. 13, 2004.

### FIELD OF THE INVENTION

The present invention relates to benzenesulfonic acid salts of clopidogrel, processes for their preparation, and their use for producing pharmaceutical formulations.

### BACKGROUND OF THE INVENTION

Clopidogrel (5-methyl α-(4,5,6,7-tetrahydro[2,3-c]thienopyridyl)(2-chlorophenyl)acetate is disclosed as active ingredient in EP-A-0 099 802. Clopidogrel acts as platelet aggregation inhibitor and can therefore be employed for example for preventing thromboembolic events such as, for example, stroke or myocardial infarction.

EP-A-0 281 459 proposes the use in pharmaceutical formulations of inorganic salts of (S)-(+)-clopidogrel, in particular (S)-(+)-clopidogrel hydrogensulfate. This document also discloses organic salts of clopidogrel, but these are described as amorphous and/or hygroscopic, and difficult to purify.

The (S)-(+)-clopidogrel hydrogensulfate employed in pharmaceutical formulations has the disadvantage that concentrated sulfuric acid is necessary to prepare it, and that corresponding products have a very strong acidic reaction because of the acidic proton. These acidic properties have an adverse effect on compatibility with many pharmaceutical excipients and thus the stability of corresponding drug forms. There is thus a need for stable forms of clopidogrel which are easy to purify and easy to process with various pharmaceutical excipients such as medicament carriers and additives.

### SUMMARY OF THE INVENTION

One object of the present invention is thus to provide clopidogrel in a form which is easy to purify, is stable, and is easy to process even on the industrial scale. It is additionally intended, as far as possible, to avoid interactions with conventional medicament carriers, additives, and processing aids.

It has now been found, surprisingly, that contrary to the disclosure in EP-A-0 281 459, the benzenesulfonic acid salt of clopidogrel is suitable under certain conditions for producing pharmaceutical formulations.

The present invention thus relates to benzenesulfonic acid salts of clopidogrel, which are at least partly in crystalline form. The present invention additionally relates to benzenesulfonic acid salts of clopidogrel, which may be prepared by precipitating the salt from a clopidogrel solution containing toluene and/or dioxane.

These salts are obtained by reacting clopidogrel with benzenesulfonic acid in 1,4-dioxane or toluene, respectively, followed by crystallisation from these solvents. The crystalline salts thus obtained are the 1,4-dioxane solvate (form I clopidogrel besylate) or the toluene solvate (form 11 clopidogrel besylate). Such solvate formation is conducive to the crystallisation of the salts. This process has turned out to be a highly effective purification operation for the active ingredient clopidogrel. Such a process, however, may result in the formation of solvates. Thus, the present invention also relates to a crystalline, substantially solvent-free benzenesulfonic acid salt of clopidogrel.

### BRIEF DESCRIPTION OF THE DRAWINGS

FIG. 1 shows the x-ray powder spectrum of a benzenesulfonic acid salt of clopidogrel precipitated from toluene, which was recorded with a STOE STADI P transmission diffractometer using copper Kα radiation.

FIG. 2 shows the x-ray powder spectrum of a benzenesulfonic acid salt of clopidogrel precipitated from dioxane, which was recorded with a STOE STADI P transmission diffractometer using copper Kα radiation.

FIG. 3 depicts the results of a stability study of clopidogrel salts under the influence of heat.

FIG. 4 shows the X-ray powder spectrum of form III crystalline clopidogrel besylate.

FIG. 5 shows the IR spectrum of form III crystalline clopidogrel besylate.

### DETAILED DESCRIPTION OF THE INVENTION

One form of the clopidogrel that can be employed according to the invention is a racemic mixture of the two clopidogrel isomers. An alternative possibility is to use the pure isomers, in which case the (S)-(+)-clopidogrel isomer is preferred.

It has surprisingly been found according to the invention that, contrary to the teaching of EP-A-0 281 459, it is possible to incorporate benzenesulfonic acid salts of clopidogrel into pharmaceutical formulations and, in particular, into pharmaceutical formulations to be administered orally. The invention thus also includes the use of benzenesulfonic acid salts of clopidogrel for producing pharmaceutical formulations, and pharmaceutical formulations comprising such salts.

A benzenesulfonic acid salt of clopidogrel according to the present invention is at least partly, preferably completely, crystalline. The crystalline or partly crystalline form of such salts may be purified more easily compared to the amorphous forms disclosed in EP-A-0 281 459. In addition, the crystalline salt forms of the present invention may be further processed to pharmaceutical formulations more easily.

It has additionally been found according to the invention that the desired and, in particular, crystalline benzenesulfonic acid salt of clopidogrel can be prepared simply and in a form which is favorable for further processing to a pharmaceutical formulation by precipitating the salt from a clopidogrel solution, which comprises toluene and/or dioxane as solvent(s). It is possible and preferred to employ mixtures of toluene and acetone or dioxane and ethyl acetate.

For example, clopidogrel base can be dissolved in toluene, and the desired salt can be precipitated by adding a benzenesulfonic acid solution in acetone. In another embodiment, both clopidogrel base and the benzenesulfonic acid can be dissolved in dioxane and mixed, and the desired salt can be precipitated by adding ethyl acetate.

The benzenesulfonic acid salt of clopidogrel of the present invention can be obtained in good yield and purity by the process described above, so that this salt is particularly suitable for producing pharmaceutical formulations, especially when it is in crystalline form.

It has additionally been found that the benzenesulfonic acid salt of clopidogrel has particularly advantageous properties for example in relation to its crystallinity when it comprises solvent molecules. The solvent molecules included in the salt as solvate are derived from the solution from which the salt has been precipitated. The solvate preferably comprises toluene or dioxane.

The benzenesulfonic acid salt of clopidogrel precipitated from toluene comprises toluene molecules. The 10 characteristic (i.e. most intense) peaks in the x-ray powder spectrum of this solvate have the following 2Θ-values:

| relative intensity | 2Θ (degrees) |
|---|---|
| 99.11 | 10.80 |
| 100.0 | 12.08 |
| 96.77 | 16.09 |
| 62.57 | 16.66 |
| 84.58 | 20.22 |
| 93.53 | 21.50 |
| 66.00 | 22.56 |
| 78.33 | 22.91 |
| 81.82 | 23.45 |
| 56.15 | 24.92 |

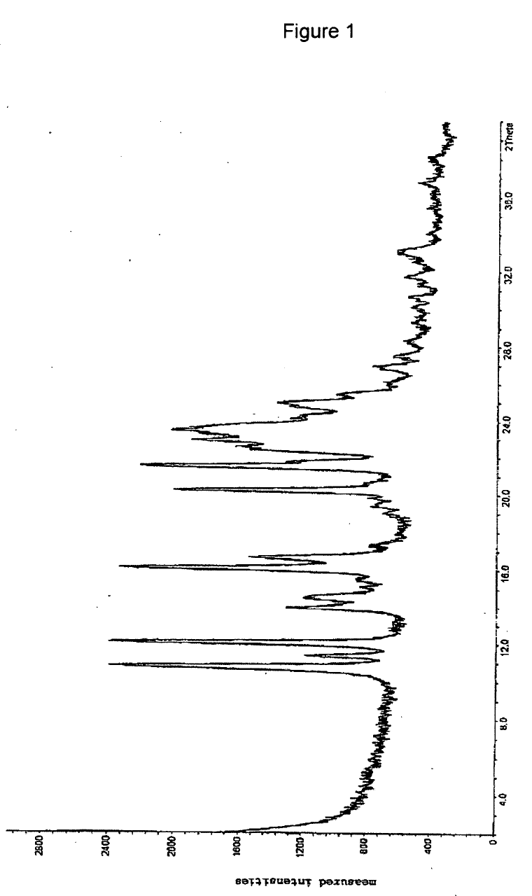

The complete x-ray powder spectrum, which was recorded with a STOE STADI P transmission diffractometer using copper Kα radiation, is depicted in FIG. 1. As is known to one skilled in the art, the identity of a crystalline sample is established with respect to a reference material if the scattering angles of the ten strongest reflections obtained for the sample agree to within +0.20 degrees with that of the reference sample. Thus, for purposes this invention, two samples are considered to be the same if the scattering angles (in degrees 2Θ) of the ten strongest reflections obtained for the respective samples are the same to within ±0.20 degrees. See e.g., “X-Ray Diffraction,” Physical Tests, <941>, The United States Pharmacopoeia, 24th Ed., United States Pharmacopeia Convention, Rockville, Md, 1999.

The benzenesulfonic acid salt of clopidogrel precipitated from dioxane comprises dioxane molecules. The 10 characteristic peaks in the x-ray powder spectrum of this solvate have the following 2Θ values:

| relative intensity | 2Θ (degrees) |
|---|---|
| 51.66 | 10.78 |
| 54.15 | 10.87 |
| 90.13 | 12.13 |
| 50.83 | 14.34 |
| 50.27 | 16.43 |
| 76.03 | 21.57 |
| 81.19 | 22.87 |
| 100.00 | 23.06 |
| 54.18 | 23.72 |
| 54.05 | 25.17 |

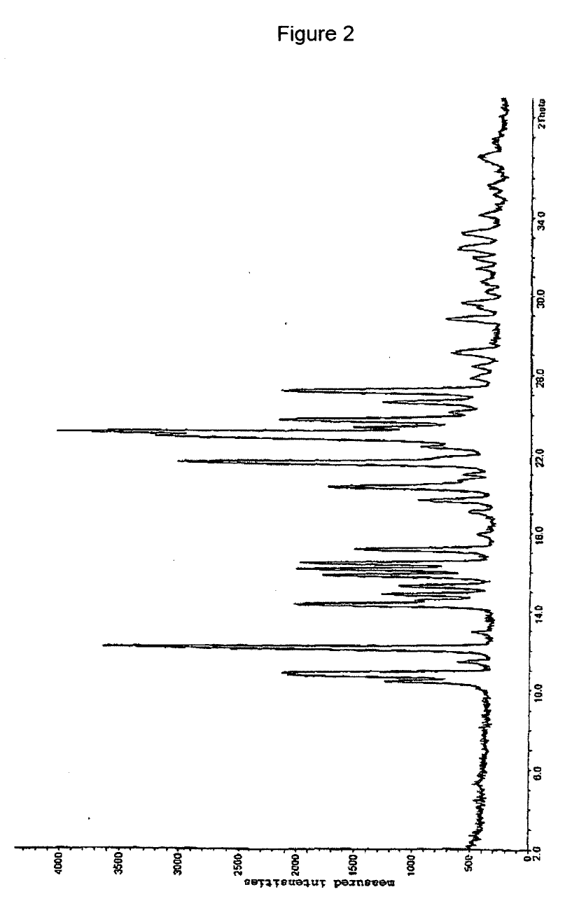

The x-ray powder spectrum of this salt, which was measured as described above, is represented in FIG. 2.

It has also been found that the benzenesulfonic acid salt of clopidogrel is obtained in particularly high purity compared with other clopidogrel salts. On crystallization from dioxane, for example, a salt with only 0.085% impurities (according to HPLC) is obtained. Such a salt is, therefore, suitable for preparing pure clopidogrel. Thus, the present invention also relates to a process for purifying clopidogrel, where impure clopidogrel or a salt thereof is, where appropriate after liberation of clopidogrel base, converted into the benzenesulfonic acid salt of clopidogrel and, if desired, subsequently clopidogrel base is liberated from the isolated salt of benzenesulfonic acid and/or is converted into another salt.

The present invention also includes benzenesulfonic acid salts of clopidogrel, which are substantially solvent free. As used herein, “substantially solvent-free” means that the salt complies with the maximum limits for residual solvents defined by the International Conference for Harmonization (Topic Q3C, Impurities: Residual Solvents (CPMP/ICH/283/95).) For example, the salt should contain less than 380 ppm, preferably less than 100 ppm, and especially preferably less than 50 ppm of residual solvent, especially 1,4-dioxane.

Such salts have been designated form III clopidogrel besylate salts. The 10 characteristic peaks in the x-ray powder spectrum of this salt have the following 2Θ values:

| relative intensity | 2Θ (degrees) |
|---|---|
| 26.31 | 10.81 |
| 26.09 | 13.58 |
| 40.66 | 14.30 |
| 100.00 | 16.26 |
| 31.85 | 17.12 |
| 31.14 | 21.00 |
| 37.54 | 21.31 |
| 31.36 | 21.83 |
| 63.79 | 23.04 |
| 27.14 | 24.44 |

To prepare the form III clopidogrel besylate salt, the 1,4-dioxane solvate molecules contained in the benzenesulfonic acid salts of clopidogrel described above may be removed under certain conditions. In our first experiments, it was not possible to remove the 1,4-dioxane at room temperature in vacuo. Only useless amorphous products whose original crystal structure had been lost were obtained. We then found, to our surprise, that the 1,4-dioxane solvate transforms with loss of 1,4-dioxane when stored for a longer period of time, possible at a slightly elevated temperature for some days, in open vessels exposed to the atmosphere, resulting in a new, 1,4-dioxane-free crystalline benzenesulfonic acid salt of clopidogrel (form III clopidogrel besylate salt). This process, however, is slow and difficult to control for technical exploitation.

It was surprising to find that the desired form III clopidogrel besylate salt may be obtained at a technically relevant scale and effectively from an economic point of view by trituration of a 1,4-dioxane-free benzenesulfonic acid salt of clopidogrel with a solvent which should also be 1,4-dioxane-free and the addition of seed crystals of form III clopidogrel besylate salt. Ethyl acetate has turned out to be a particularly suitable solvent for triturating the salt. Alternatively, the form III clopidogrel besylate salt may be obtained by dropping a solution of benzenesulfonic acid in a suitable solvent, e.g., ethyl acetate, into a solution of clopidogrel in a suitable solvent, e.g., mixtures of acetone and ethyl acetate, and adding seed crystals of form III clopidogrel besylate salt.

The seed crystals of form III clopidogrel besylate salt needed for these processes may be prepared by precipitating a benzenesulfonic acid salt of clopidogrel from a 1,4-dioxane-containing solvent. The 1,4-dioxane solvate thus obtained is then stored while exposed to the atmosphere as described above until a 1,4-dioxane-free benzenesulfonic acid salt of clopidogrel is obtained. As a result of this kind of storage, the 1,4-dioxane solvate is converted into the desired form III clopidogrel besylate salt.

The 1,4-dioxane-free benzenesulfonic acid salt of clopidogrel used as a feedstock in the method of the invention may be obtained by concentrating a solution containing benzenesulfonic acid and clopidogrel. This solution should be substantially free of 1,4-dioxane. The salt obtained as a result of concentrating a suitable solution is usually amorphous at the beginning and then crystallizes during trituration to the desired form III clopidogrel besylate salt when the necessary seed crystals are added.

The method of the invention for preparing form III clopidogrel besylate salt preferably comprises the steps of precipitating a benzenesulfonic acid salt of clopidogrel from a 1,4-dioxane-containing solvent and dissolving the 1,4-dioxane-solvate thus obtained in a substantially 1,4-dioxane-free solvent, followed by evaporation of the solvent. Evaporation may be carried out at a reduced pressure and, optionally, at an elevated temperature. Suitable substantially 1,4-dioxane-free solvents are alcohols, ketones, or hydrocarbons, especially ethanol, isopropanol, 1-propanol, and acetone. If necessary, the steps of dissolution and evaporation may be carried out repeatedly, for example twice, using the same or different solvents.

It has been shown that, by this method, the 1,4-dioxane may be removed to a practically quantitative extent despite its comparatively high boiling point of 101.3° C. Surprisingly, the method also works with solvents having a much lower boiling point than 1,4-dioxane, such as ethanol (78.5° C.), isopropanol (82.4° C.), 1-propanol (97.4° C.) and acetone (56.5° C.).

The resulting product is free or at least substantially free of 1,4-dioxane (for example <50 ppm 1,4-dioxane) and highly crystalline. It has a melting point of 135° C. to 138° C.

The form III clopidogrel besylate of the invention may be incorporated easily into pharmaceutical formulations. Thus, the invention also encompasses this new form of clopidogrel besylate for preparing pharmaceutical formulations, and pharmaceutical formulations containing this form.

A further aspect of the present invention comprises provision of the benzenesulfonic acid salts of clopidogrel, including form III clopidogrel besylate salt, in a form which can be further processed easily. This is achieved according to the invention by applying the salt to a solid adsorbent. This results in active ingredient particles, which can easily be poured and metered.

A suitable adsorbent is any physiologically and pharmaceutically acceptable, preferably particulate, solid which is able to adsorb the salts of the present invention. The solid is preferably a free-flowing powder which can easily be processed further to oral pharmaceutical formulations.

Physiologically and pharmaceutically acceptable adsorbents are, for example:
1. natural or prepared adsorbents from the group of aluminas (clay materials).and other earths and minerals, e.g. attapulgites, aluminum magnesium silicates (Carrisorb®, Gelsorb®), magnesium aluminum silicates (Pharamsorb®, Veegum®), magnesium silicates (talc), calcium silicates, bentonites, kaolin, magnesium trisilicates, montmorillonites, china clays (bole), sepiolites (meerschaum)
2. silica gels, kieselguhr, silicas
3. colloidal (anhydrous) silicas (hydrophobic or hydrophilic Aerosils®, Cab-o-sils®)
4. celluloses, modified celluloses, finely crystalline and microcrystalline celluloses, and cellulose derivatives, cellulose acetate, cellulose fatty acid esters, cellulose nitrates, cellulose ethers (carboxymethylcelluloses, ethylcelluloses, hydroxyethylcelluloses, hydroxypropylcelluloses, methylcelluloses, methylethyl-celluloses, methylhydroxypropylcelluloses)
5. sugars and sugar derivatives (mono- and polysaccharides), lactoses, dextrans, dextrose, cyclodextrins
6. native corn, rice, cassava, wheat, potato starches and derivatives thereof, dextrins, pregelatinized, wholly or partly hydrolyzed starches
7. solid polyols, especially mannitol or sorbitol
8. polyacrylates, acrylic acid polymers and copolymers
9. phosphates, sulfates, carbonates, gluconates, oxides of alkali metals and alkaline earth metals, and physiologically acceptable heavy metals and transition metals
10. guar flour, guar gum
11. locust bean gum (carob flour, carob gum)
12. alginic acid, alginates and seaweed flour
13. tragacanth
14. vegetable carbon (charcoal)
15. pectins and amylopectins
16. N-vinylpyrrolidone polymers such as, for example, povidone or crospovidone.

The adsorbents can be employed singly or in a mixture of two or more adsorbents. An additional possibility is for the active ingredient particles of the invention to comprise besides the adsorbent conventional pharmaceutical excipients, for example for producing direct tabletting mixtures or for producing granules for further processing to medicaments. An alternative possibility is for the active ingredient particles of the invention to be mixed after production thereof with appropriate excipients and then be further processed to pharmaceutical formulations.

Particularly preferred adsorbents are lactose (e.g. Lactopress®), mannitol (e.g. Mannogem®) and cellulose (e.g. Celphere®), especially lactose. Granules based on pyrogenic silica are preferably not employed as adsorbent, although this is possible.

Desorption can be controlled by employing suitable wetting agents. The stability can be improved by adding, for example, antioxidants such as, for example, ascorbic acid and salts thereof. Further suitable aids are emulsifiers, solvents and solubilizers.

The active ingredient particles of the invention can be obtained for example from a solvent in which the adsorbent is insoluble, slightly soluble, or partly soluble and the benzenesulfonic acid salts of clopidogrel, including the form III clopidogrel besylate salt, are soluble. The adsorbent can be suspended in the solvent for this purpose. Before or after the suspension step, the benzenesulfonic acid salt of clopidogrel can be dissolved in the solvent. The active ingredient can, in this case, be added either directly or as solution in the same or another solvent. Subsequently, the active ingredient particles, which comprise the salt applied to the adsorbent are obtained from the solvent, for example by evaporating the solvent.

Suitable solvents include conventional solvents in which the chosen adsorbent is not soluble, slightly soluble, or partly soluble and the benzenesulfonic acid salt of clopidogrel is soluble. For example, the solvents described above for preparing the salt can be used. An alternative possibility is to employ, for example, diethyl ether or methyl tert-butyl ether.

In an alternative embodiment of the process of the invention for preparing active ingredient particles, the last stage of the synthesis of clopidogrel is carried out in the presence of the adsorbent. It is possible in this way to prepare the desired active ingredient particles without an isolating intermediate step. It is also possible, for example, to mix clopidogrel and benzenesulfonic acid with the suspension of the adsorbent. In this case, the clopidogrel and the benzenesulfonic acid can each be dissolved separately in a solvent and added simultaneously or successively to the suspension. An alternative possibility is for the clopidogrel and the benzenesulfonic acid to be added in pure form to the suspension. Individual ingredients can also be premixed separately and then added together to the suspension.

The weight ratio of adsorbent to benzenesulfonic acid salt of clopidogrel adsorbed thereon is not particularly important for the present invention and can be selected by the skilled worker depending on the desired purpose of use. However, on further processing to oral pharmaceutical formulations, care should be taken that sufficient clopidogrel is applied to the adsorbent for the desired dosage in the unit dose form to be reached. For example, the weight ratio of benzenesulfonic acid salt of clopidogrel, based on free clopidogrel base, to adsorbent can be in the range from 2:1 to 1:6, i.e. for example 1 part by weight of clopidogrel base to 6 parts by weight of adsorbent, preferably in the range from 1:1 to 1:3.

The following examples are provided to further illustrate the compounds, compositions, and processes of the present invention. These examples are illustrative only and are not intended to limit the scope of the invention in any way.

### EXAMPLES

In examples 1-8, the x-ray powder spectra were recorded with a STOE STADI P transmission diffractometer with copper Kα radiation, the NMR data were recorded with a Varian Unityplus 300 instrument, and the CHN data were recorded with a Carlo Erba 1106 analyzer.

### Example 1

### Preparation of Clopidogrel Benzenesulfonate from Acetone/Toluene

4.0 g (12.5 mmol) of clopidogrel base were dissolved in 30 ml of toluene, and 2.0 g (12.5 mmol) of anhydrous benzenesulfonic acid in 10 ml of acetone were added thereto. After some time and scratching with a glass rod, the product solidified and was filtered off with suction. The product was dried overnight in a desiccator attached to a vacuum pump.

| | |
|---|---|
| Yield: 67% | m.p. 87°-90° C. |

NMR(ppm): 2.35 (toluene), 3.0-3.5 and 3.8-4.3(4H), 3.79 (3H), 4.8-5.2 (1H), 5.69 (1H), 6.6-6.8 (1H), 7.2-8.0 (12H)

The x-ray powder spectrum of the salt is represented in FIG. 1.

On further drying until the toluene was completely removed from the salt, the crystal structure collapsed and amorphous clopidogrel benzenesulfonate was obtained.

### Example 2

### Preparation of Clopidogrel Benzenesulfonate From Dioxane

A solution of 53.7 g (339.7 mmol) of anhydrous benzenesulfonic acid in 100 ml of dioxane were added while stirring to 109.2 g (339.7 mmol) of clopidogrel base dissolved in 300 ml of dioxane at 10° C. 250 ml of ethyl acetate were added to this solution, and this solution was placed in a deep freeze overnight. The solution was allowed to warm to room temperature, and the residue was filtered off with suction and washed with ethyl acetate. The product was dried in vacuo at room temperature for 48 h.

| | |
|---|---|
| Yield: 71% | m.p. 93°-95° C. |

### Elemental Analysis:

| | | |
|---|---|---|
| | calculated for clopidogrel | |
| Values (%) | besylate *½ dioxane | found |

| | | | |
|---|---|---|---|
| C | 55.01 | 55.28 | 55.03 |
| H | 5.00 | 5.12 | 4.99 |
| N | 2.67 | 2.62 | 2.53 |

NMR (ppm): 3.0-3.5 and 3.8-4.3 (4H), 3.79 (3H), 4.8-5.2 (1H), 5.68-5.72 (1H), 6.6-6.8 (1H), 7.2-8.0 (12H), 3.70 (4H; 1/2 dioxane)

The x-ray powder spectrum of this salt is represented in FIG. 2.

### Example 3

### Stability Investigations

3.1 The stability of various clopidogrel salts was investigated under a plurality of conditions. The salts employed were form III of clopidogrel hydrogensulfate, clopidogrel hydrochloride (prepared as disclosed in EP 281 459), amorphous clopidogrel benzenesulfonate, and crystalline clopidogrel benzenesulfonate (from Example 2 above). The following tests were carried out:

### Stability Under Acidic Conditions

50 mg of the respective salts were separately weighed into volumetric flasks (100 ml), and 2 ml of 1N HCl were added. The flasks were then stored either at room temperature for 5 h or at 80° C. for 5 h. After the end of the particular experiment and, where appropriate, cooling to room temperature, 2 ml of 1N NaOH were added to each flask, and the volume was made up to 100 ml with the mobile phase.

The total impurities relative to the clopidogrel base were determined by HPLC and summarized in Tables 14 below.

### Stability Under Basic Conditions

50 mg of the respective salts were separately weighed into volumetric flasks (100 ml), and 2 ml of 1N NaOH were added. The flasks were then stored either at room temperature for 5 h or at 80° C. for 5 h. After the end of the particular experiment and, where appropriate, cooling to room temperature, 2 ml of 1N HCl were added to each flask, and the volume was made up to 100 ml with the mobile phase.

The total impurities relative to the clopidogrel base were determined by HPLC and summarized in Tables 1-4 below.

### Stability Under Oxidative Conditions

50 mg of the respective salts were separately weighed into volumetric flasks (100 ml), and 2 ml of 3% H₂O₂ are added. The flasks were then stored either at room temperature for 5 h or at 80° C. for 5 h. After the end of the particular experiment and, where appropriate, cooling to room temperature, the volume in each flask was made up to 100 ml with the mobile phase.

The total impurities relative to the clopidogrel base were determined by HPLC and summarized in Tables 1-4 below.

### Stability Under Neutral Conditions

50 mg of the respective salts were weighed separately into volumetric flasks (100 ml), and 2 ml of water were added. The flasks were then stored either at room temperature for 5 h or at 80° C. for 5 h. After the end of the particular experiment and, where appropriate, cooling to room temperature, the volume in each flask was made up to 100 ml with the mobile phase.

The total impurities relative to the clopidogrel base were determined by HPLC and summarized in Tables 1-4 below.

### Stability Under the Influence of Heat

50 mg of the respective salts were weighed separately into volumetric flasks (100 ml) and stored at 80° C. for 20 h. After the end of the particular experiment and cooling to room temperature, the volume in each flask was made up to 100 ml with the mobile phase.

The results were determined by HPLC and summarized in Tables 1-4 below.

The HPLC measurements took place in all cases under the following conditions with UV detection:

| | |
|---|---|
| Column: | Hypersil BDS 5 μm, 250 * 4.6 mm |
| Mobile phase: | methanol 650 ml |
| | 0.05 M 1-octanesulfonic acid Na salt 350 ml |
| | (adjusted to pH 2.5 with triethylamine and |
| | phosphoric acid). |
| Flow rate: | 1 ml/min |
| Column temperature: | room temperature |
| Wavelength: | 215 nm |
| Injection volume: | 20 μl |
| Retention time: | approx. 15 min |

### Clopidogrel Hydrogensulfate

TABLE 1

| Condition | Room temperature | 80° C. |
|---|---|---|
| acidic | 0.32% | 2.96% |
| Alkaline | 0.32% | 59.48% |
| oxidizing | 0.33% | 3.50% |
| Neutral | 0.40% | 1.63% |
| Heat | — | 0.31% |

### Clopidogrel Hydrochloride

TABLE 2

| Condition | Room temperature | 80° C. |
|---|---|---|
| Acidic | 1.86% | 3.31% |
| Alkaline | 1.86% | 72.89% |
| oxidizing | 1.83% | 4.16% |
| Neutral | 1.84% | 4.33% |
| heat | — | 32.43% |

### Clopidogrel Benzenesulfonate (Amorphous)

TABLE 3

| Condition | Room temperature | 80° C. |
|---|---|---|
| acidic | 0.64% | 2.36% |
| Alkaline | 0.64% | 25.04% |
| oxidizing | 0.83% | 2.94% |
| Neutral | 0.85% | 3.01% |
| Heat | — | 11.52% |

### Clopidogrel Benzenesulfonate (Crystalline)

TABLE 4

| Condition | Room temperature | 80° C. |
|---|---|---|
| Acidic | 0.14% | 2.76% |
| Alkaline | 0.14% | 28.05% |
| oxidizing | 0.13% | 3.98% |
| Neutral | 0.19% | 4.18% |
| Heat | — | 4.52% |

It is evident that, contrary to the teaching of EP 281 459, the stability of amorphous clopidogrel benzenesulfonate is comparable to and, especially under alkaline conditions, is considerably higher than that of the hydrogensulfate and hydrochloride salts of clopidogrel. In addition, the stability of the crystalline form of clopidogrel benzenesulfonate is increased further compared with the amorphous form of this salt, in particular at room temperature, which is important for the storage of pharmaceutical products. Crystalline clopidogrel benzenesulfonate is, in fact, more stable than clopidogrel hydrogensulfate, which is employed in pharmaceutical formulations.

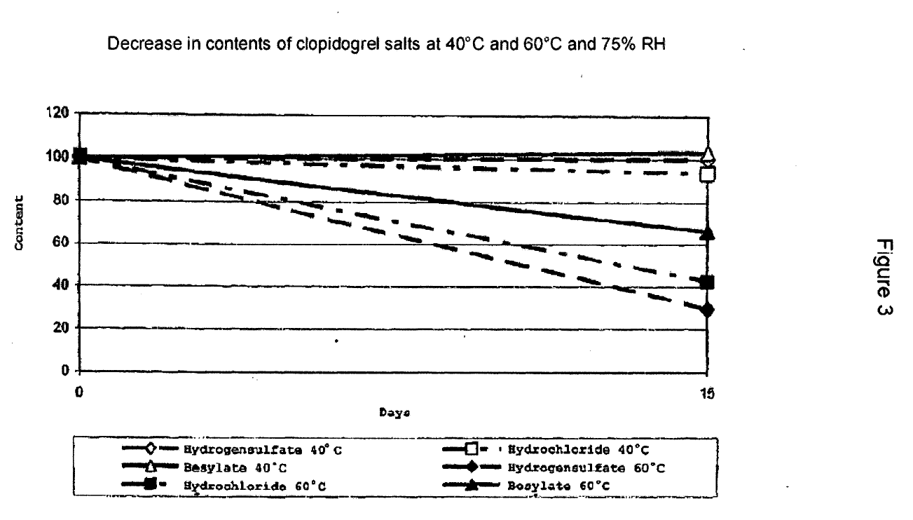

3.2 In addition, the decrease in the contents of clopidogrel hydrogensulfate, hydrochloride and besylate (crystalline) was investigated at 40° and 60° C. and 75% relative humidity for 15 days. The results are represented in FIG. 3.

It is evident that the best stability is shown by the besylate salt (clopidogrel benzenesulfonate) both at 40° and at 60° C.

### Example 4

### Adsorbate of (S)-(+)-clopidogrel Besylate on Calcium Gluconate as Carrier Material

A solution of 11 g (69.5 mmol) of anhydrous benzenesulfonic acid in 100 ml of cold, anhydrous diethyl ether was slowly added dropwise (approx. 30 min) to a vigorously stirred solution of 19.7 g (61.4 mmol) of (S)-(+)-clopidogrel in 300 ml of anhydrous diethyl ether at 3° C. A prepared suspension of 28 g of calcium gluconate in cold, anhydrous diethyl ether was then slowly added. The resulting adsorbate was filtered off with suction, washed with ice-cold, anhydrous diethyl ether and then dried.

A white, free-flowing powder was obtained.

### Example 5

### Adsorbate of (S)-(+)-clopidogrel Besylate on Silica Gel/Mannitol as Carrier Material

20 g (62.3 mmol) of (S)-(+)-clopidogrel and 11 g (69.5 mmol) of anhydrous benzenesulfonic acid were reacted in 200 ml of anhydrous diethyl ether at a temperature of 2°-3° C. A suspension of 2 g of silica and 20 g of mannitol in 100 ml of anhydrous diethyl ether was then slowly added. The resulting adsorbate was filtered off with suction in the cold, washed with ice-cold, anhydrous diethyl ether and then dried.

39 g of a white, free-flowing powder were obtained.

### Example 6

Two different processes for preparing adsorbates of clopidogrel salts were used. In the first process, the salt was dissolved in a suitable solvent, and the adsorbent was suspended in this solution.

In a second series of tests, clopidogrel base was dissolved in a suitable solvent, the adsorbent was added, and the salt was precipitated onto the carrier material.

The adsorbents employed in each of the tests were lactose (Lactopress®), mannitol (Mannogem®) and cellulose (Celphere®).

The following tests were carried out:

### Clopidogrel Besylate Adsorbates with Isolation of the Salt

1.5 g (3.1 mmol) of clopidogrel besylate were dissolved in 20 ml of acetone, and 1.5 g of adsorbent were added. The solvent was stripped off, and the residue was briefly suspended in MTB ether and then dried in vacuo.

### Clopidogrel Besylate Adsorbates Without Previous Isolation of the Salts

### 1. Diethyl Ether as Solvent

4.018 g (12.5 mmol) of clopidogrel base were dissolved in 20 ml of diethyl ether. 6 g of adsorbent and 1.977 g (12.5 mmol) of benzenesulfonic acid were added in 20 ml of ether. The solid product was filtered off with suction, washed with ether and dried in vacuo.

### 2. Methyl Tert-Butyl Ether (MTB Ether) as Solvent

4.018 g (12.5 mmol) of clopidogrel base were dissolved in 40 ml of MTB ether. 6 g of adsorbent and 1.977 g (12.5 mmol) of benzenesulfonic acid were added in 50 ml of MTB ether. The solid product was filtered off with suction, washed with MTB ether and dried in vacuo.

### Example 7

The stability of the adsorbates obtained as in Example 6 was investigated. The adsorbates remained powdery at room temperature and did not change in color over more than two months.

The decrease in the active ingredient content on storage at 40° or 60° C. and 75% relative humidity for 15 days was measured. The results are summarized in Table 5 below (content after 15 days (initial level standardized at 100%)).

TABLE 5

| Clopidogrel besylate | 40° C. | 60° C. |
|---|---|---|
| Pure (free) salt | 103.32 | 66.48 |
| Lactopress/diethyl ether | 106.91 | 94.47 |
| Lactopress/MTB ether | 94.74 | 92.58 |

It is evident that the adsorbates show a greater stability at elevated temperature compared with the free salt.

### Example 8

Adsorbates prepared as in Example 6 can be compressed directly into tablets. This is made clear by the following exemplary formulations. The amount used of the further excipients indicated in the following examples are known to the skilled worker through his basic knowledge and can be found in standard works on the formulation of tablets such as, for example, Ritschel et al., “Die Tablette”, Editio Cantor—Aulendorf, 2^{nd} ed., 2002, and “Remington: The Science and Practice of Pharmacy”, 20^{th} ed. (2003).

### a) Clopidogrel Besylate-Microcrystalline Cellulose Adsorbate

Clopidogrel tablets with a total mass of 275 mg were produced from the adsorbate by direct compression with the following composition:

| | |
|---|---|
| Clopidogrel besylate-microcrystalline cellulose adsorbate | 219.54 mg |
| (equivalent to 75 mg of clopidogrel base) | |
| Excipients (lubricant, fillers, disintegrant, flow regulator, | ad 275 mg |
| wetting agent) | |

Properties of the mixture ready for compression and of the tablets:

| | |
|---|---|
| Compressibility and flowability: | satisfactory to good |
| Average hardness: | 101 N |
| Friability: | 0.11% |
| Disintegration time: | 65 sec |
| Release: | 100% after 30 min. |

The tablets obtained in this way can also be provided with a coating such as, for example, an enteric coating, a taste-masking coating, or a color coating.

### b) Clopidogrel Besylate-Mannitol Adsorbate

Clopidogrel tablets with a total mass of 275 mg were produced from the adsorbate by direct compression with the following composition:
Clopidogrel besylate-mannitol adsorbate (equivalent to 219.54 mg 75 mg of clopidogrel base)
Excipients (lubricant, fillers, disintegrant, flow regulator, ad 275 mg wetting agent)

Properties of the mixture ready for compression and of the tablets:

| | |
|---|---|
| Compressibility and flowability: | satisfactory to good |
| Average hardness: | 106 N |
| Friability: | 0.15% |
| Disintegration time: | 62 sec |
| Release: | 100% after 30 min. |

The tablets obtained in this way can be provided with a coating such as, for example, an enteric coating, a taste-masking coating, or a color coating.

### c) Clopidogrel Besylate-Lactose Adsorbate

Clopidogrel tablets with a total mass of 275 mg were produced from the adsorbate by direct compression with the following composition:
Clopidogrel besylate-lactose adsorbate (equivalent to 75 mg 219.54 mg of clopidogrel base)
Excipients (lubricant, fillers, disintegrant, flow regulator, ad 275 mg wetting agent)

Properties of the mixture ready for compression and of the tablets:

| | |
|---|---|
| Compressibility and flowability: | satisfactory to good |
| Average hardness: | 96 N |
| Friability: | 0.21% |
| Disintegration time: | 76 sec |
| Release: | 100% after 30 min. |

The tablets obtained in this way can be provided with a coating such as, for example, an enteric coating, a taste-masking coating, or a color coating.

### Example 9

### Form III Crystalline Clopidogrel Besylate

In this example, the x-ray powder spectra were recorded by means of a STOE STADI P transmission diffractometer with copper-K_{α} radiation and the NMR data with a Varian UNITYplus 500 device. The IR spectrum was recorded as a KBr compact by means of an IR spectrum One FTIR spectrometer by Perkin-Elmer.

1.9 g of clopidogrel besylate·1,4-dioxane (prepared according to Example 2) were dissolved in 15 ml of ethanol and the solvent was distilled off on a rotary evaporator at a pressure of 12 mbar. The amorphous residue was then dissolved in 15 ml of acetone and distilled off again at 12 mbar (temperature of the bath: 32° C.). The amorphous residue was triturated in 20 ml of ethyl acetate and seeded with form III clopidogrel besylate. The resulting crystals were removed by suction and washed with 5 ml of ethyl acetate.

Yield: 1.7 g, melting point 135-138° C. Residual 1,4-dioxane content: <50 ppm.

¹H-NMR (ppm) CDCl₃: 3.0-4.3 (m, 5H); 3.79 (s, 3H, OCH₃); 4.8-5.2 (m, 1H); 5.7 (m, 1H); 6.6-6.8 (d, 1H); 7.2-8.0 (m, 10H); 12.5 (broad, 1H).

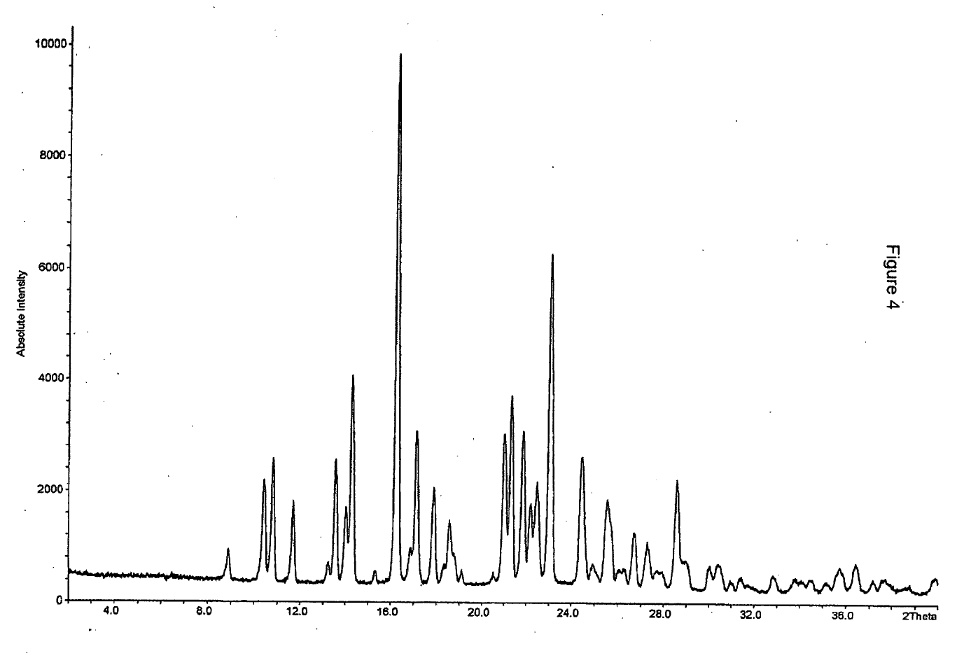

The x-ray powder spectrum of this salt is shown in FIG. 4.

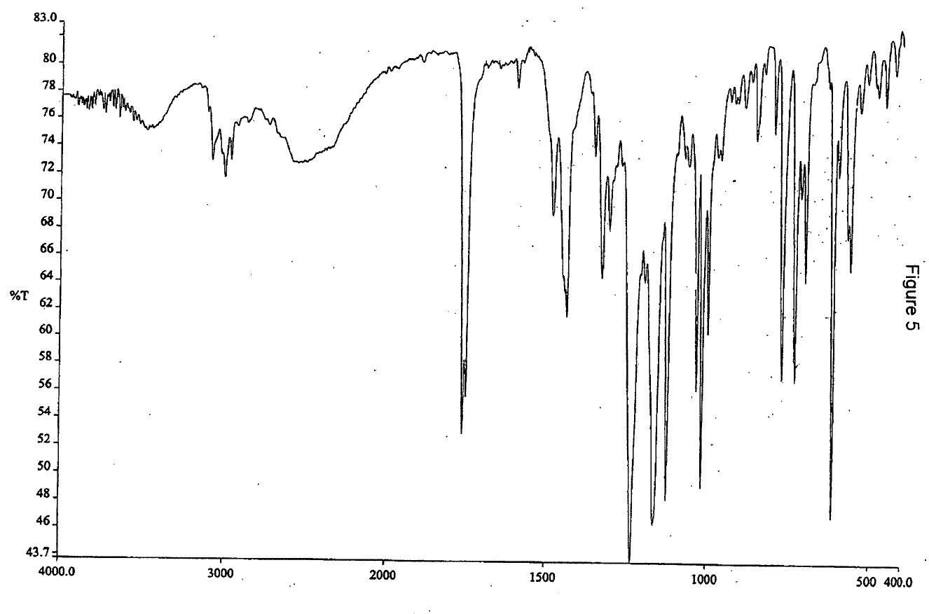

The IR spectrum of this salt is shown in FIG. 5.

IR (KBr) cm⁻¹: 3839, 3752, 3736, 3690, 3672, 3650, 3487, 3078, 2999, 2961,2543,1759,1750, 1592, 1479,1434, 1349, 1324, 1302, 1232, 1192, 1162, 1123, 1058, 1032, 1015, 995, 959, 931, 917, 885, 848, 792, 766, 727, 709, 695, 612, 595, 566, 558, 530, 509, 478,453, 424.

The invention being thus described, it will be obvious that the same may be varied in many ways. Such variations are not to be regarded as a departure from the spirit and scope of the invention and all such modifications are intended to be included within the scope of the following claims.

1. A benzenesulfonic acid salt of clopidogrel at least partially in crystalline form.

2. A crystalline benzenesulfonic acid salt of clopidogrel.

3. A pharmaceutical composition comprising the salt of claims 1 or 2.

4. A solvate comprising the salt of claims 1 or 2.

5. A pharmaceutical composition comprising the solvate of claim 4.

6. A solvate according to claim 4 wherein the solvent portion of the solvate is selected from the group consisting of toluene and dioxane.

7. A solvate according to claim 6 having an x-ray powder spectrum comprising peaks at 10.80, 12.08, 16.09, 16.66, 20.22, 21.50, 22.56, 22.91, 23.45, and 24.92 degrees 2Θ.

8. A solvate according to claim 6 having an x-ray powder spectrum comprising peaks at 10.78, 10.87, 12.13, 14.34, 16.43, 21.57, 22.87, 23.06, 23.72, and 25.17 degrees 2Θ.

9. A unit dosage form comprising a pharmaceutically effective amount of the salt of claims 1 or 2.

10. A benzenesulfonic acid salt of clopidogrel prepared by a process comprising precipitating a solution comprising clopidogrel and a solvent selected from the group consisting of toluene, dioxane, and mixtures thereof.

11. A salt according to claim 10, which is at least partially crystalline.

12. An active ingredient particle comprising a benzenesulfonic acid salt of clopidogrel adsorbed onto a solid adsorbent.

13. A pharmaceutical composition comprising an active ingredient particle according to claim 12.

14. A unit dosage form comprising a pharmaceutically effective amount of the active ingredient particle of claim 12.

15. A process for preparing a benzenesulfonic acid salt of clopidogrel comprising precipitating a solution comprising clopidogrel and a solvent selected from the group consisting of toluene, dioxane, and mixtures thereof.

16. A process for purifying clopidogrel comprising:
(a) converting clopidogrel into its benzenesulfonic acid salt;
(b) optionally separating a clopidogrel base, if formed, from the salt; and
(c) optionally converting the clopidogrel base into another salt.

17. A process for producing an active ingredient particle comprising:
(a) precipitating from a solution comprising a benzenesulfonic acid salt of clopidogrel, a solvent, and an adsorbent an active ingredient particle comprising a benzenesulfonic acid salt of clopidogrel adsorbed to the adsorbent; and
(b) separating the active ingredient particle from the solvent.

18. The process according to claim 17, wherein the adsorbent is insoluble or slightly soluble in the solvent and the salt is soluble in the solvent.

19. The process according to claim 17 further comprising:
(a) prior to the precipitation step
(i) suspending the adsorbent in the solvent; and
(ii) dissolving the salt in the solvent.

20. The process according to claim 17 wherein the separating step comprises evaporating the solvent to obtain the active ingredient particle.

21. A process for producing an active ingredient particle comprising:
(a) combining clopidogrel and benzenesulfonic acid with an adsorbent to form a mixture; and
(b) separating from the mixture an active ingredient particle comprising a benzenesulfonic acid salt of clopidogrel adsorbed onto the adsorbent.

22. The process according to claim 21 further comprising dissolving the clopidogrel and benzenesulfonic acid in dioxane prior to combining with the adsorbent.

23. The process according to claim 21 further comprising dissolving the clopidogrel in toluene and dissolving the benzenesulfonic acid in acetone and adding the respective solutions together or serially to the adsorbent.

24. Clopidogrel besylate in form III, which form has an x-ray powder spectrum comprising peaks at 10.81, 13.58, 14.30, 16.26, 17.12, 21.00, 21.31, 21.83, 23.04, and 24.44 degrees 20Θ

25. A pharmaceutical composition comprising the clopidogrel besylate of claim 24.

26. A unit dosage form comprising a pharmaceutically effective amount of the clopidogrel besylate of claim 24.

27. A process for preparing clopidogrel besylate in form III comprising:
(a) triturating a 1,4-dioxane-free benzenesulfonic acid salt of clopidogrel with a solvent; and
(b) seeding the mixture formed in a) with crystals of clopidogrel besylate in form III to form clopidogrel besylate in form III, which form has an x-ray powder spectrum comprising peaks at 10.81, 13.58, 14.30, 16.26, 17.12, 21.00, 21.31, 21.83, 23.04, and 24.44 degrees 20.

28. A process according to claim 27, wherein the solvent is ethyl acetate.

29. A process according to claim 27 or 28 comprising, prior to step a), forming the 1,4-dioxane-free benzenesulfonic acid salt of clopidogrel by concentrating a solution comprising benzenesulfonic acid and clopidogrel.

30. A process according to claim 29, wherein the solution is substantially 1,4-dioxane-free.

31. A process according to claim 29 comprising:
a) precipitating a benzenesulfonic acid salt of clopidogrel from a solution containing clopidogrel base, benzenesulfonic acid, and 1,4-dioxane;
b) dissolving the 1,4-dioxane solvate thus obtained in a substantially 1,4-dioxane-free solvent; and
c) evaporating the solvent.

32. A process according to claim 31, wherein the solvent is selected from the group consisting of alcohols, ketones, and hydrocarbons.

33. A process according to claim 31 wherein the solvent is selected from the group consisting of ethanol, isopropanol, 1-propanol, and acetone.
